(19) United States
(12) Patent Application Publication
(10) Pub. No.: US 2006/0135715 A1
(43) Pub. Date: Jun. 22, 2006

(54) Trifluorostyrene containing compounds, and their use in polymer electrolyte membranes

(76) Inventor: Zhen-Yu Yang, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/560,878

(22) PCT Filed: Jun. 25, 2004
(86) PCT No.: PCT/US04/20706

§ 371 Date: Dec. 13, 2005

Related U.S. Application Data
(60) Provisional application No. 60/482,928, filed on Jun. 27, 2003.

(51) Int. Cl. C08F 12/30; C07C 309/29

(52) U.S. Cl. 526/243; 562/825

(57) ABSTRACT

A monomer having the structure (I) wherein R_{F} is linear or branched perfluoroalkene group, optionally containing oxygen or chlorine; and n is 1 or 2. These monomers are used in the preparation of homopolymers and copolymers that are useful in preparing polymer electrolyte membranes. Electrochemical cells, such as fuel cells, containing these membranes are also described.

### FIELD OF THE INVENTION

The present invention relates to a novel compound and its use in electrochemical cells as an electrolyte, and more particularly to the use of the compound as an electrolyte in fuel cells.

### BACKGROUND OF THE INVENTION

Electrochemical cells, such as fuel cells and lithium-ion batteries are known. Depending on the operating conditions, each type of cell places a particular set of requirements upon the electrolytes used in them. For fuel cells, this is typically dictated by the type of fuel, such as hydrogen or methanol, used to power the cell and the composition of the membrane used to separate the electrodes. Proton-exchange membrane fuel cells, powered by hydrogen as the fuel, could be run at higher operating temperatures than currently employed to take advantage of lower purity feed streams, improved electrode kinetics, better heat transfer from the fuel cell stack to improve its cooling. Waste heat is also employed in a useful fashion. However, if current fuel cells are to be operated at greater than 100° C. then they must be pressurized to maintain adequate hydration of typical proton-exchange membranes, such DuPont Nafion® perfluorosulfonic acid membrane, to support useful levels of proton conductivity.

There is an ongoing need to discover novel electrolytes that improve the performance of the latest generation of electrochemical cells, such as fuel cells and lithium-ion batteries, to develop new membrane materials that will maintain adequate proton conductivity at lower levels of hydration.

### SUMMARY OF THE INVENTION

In a first aspect, the invention provides a monomer having the following structure: 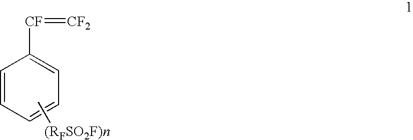
wherein R_{F} is linear or branched perfluoroalkene group, optionally containing oxygen or chlorine; and

n is 1 or 2.

In a second aspect, the invention provides a homopolymer having the following structure: 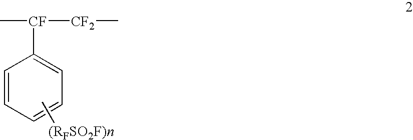
wherein R_{F} is linear or branched perfluoroalkene group, optionally containing oxygen or chlorine,

n is 1 or 2, and

In a third aspect, the invention provides a copolymer selected from the group consisting of:

(a) a copolymer having the structure: 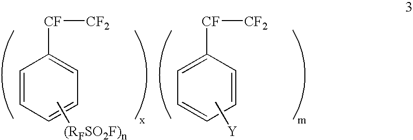
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

n is 1 or 2,

m and x are mole fractions wherein m is 0.01 to 0.99 and

x is 0.99 to 0.01; and

x+m=1

(b) a copolymer having the structure: 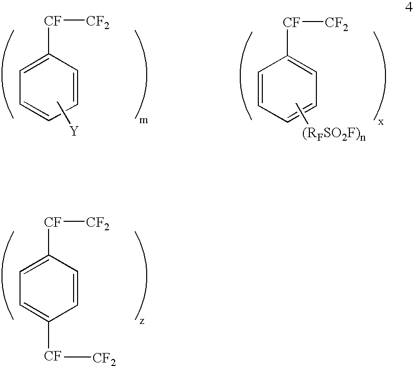
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

n is 1 or 2,

m, x and z are mole fractions wherein m is 0.01 to 0.99,

x is 0.99 to 0.01, and

z is 0.0001 to 0.10

m+x+z=1.

In a fourth aspect, the invention provides a polymer electrolyte membrane prepared from a homopolymer or copolymer selected from the group consisting of:

(a) a homopolymer having the structure: 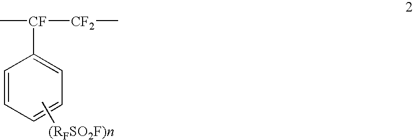
wherein R_{F} is linear or branched perfluoroalkene group, optionally containing oxygen or chlorine,

n is 1 or 2;

(b) a copolymer having the structure: 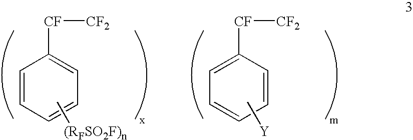
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

n is 1 or 2,

m and x are mole fractio wherein m is 0.01 to 0.99 and

x is 0.99 to 0.01; and

x+m=1; and

(c) a copolymer having the structure: 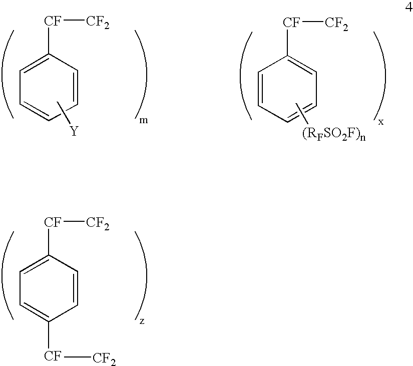
wherein R_{F} is linear or branched perfluoroalkene group, optionally containing oxygen or chlorine, Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C₁₀ carbon atoms,

n is 1 or 2,

m, x and z are mole fraction wherein m is 0.01 to 0.99,

x is 0.99 to 0.01, and

z is 0.0001 to 0.10

m+x+z=1; and mixtures thereof.

In the fourth aspect, the invention provides a polymer electrolyte membrane selected from the group consisting of:

(a) a membrane having the chemical structure: 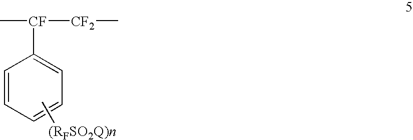
wherein R_{F} is linear or branched perfluoroalkene group, optionally containing oxygen or chlorine,

Q=OM, OH, NHSO2R_{F}, wherein M=Li⁺, Na, K or Cs,

n=1 or 2;

(b) a membrane having the chemical structure:

wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

Q=OM, OH, NHSO2R_{F}, wherein M=Li⁺, Na⁺, K⁺ or Cs⁺,

n is 1 or 2,

m and x are mole fractions wherein m is 0 to 0.99,

x is 1 to 0.001, and

x+m=1; and

(c) a membrane having the chemical structure: 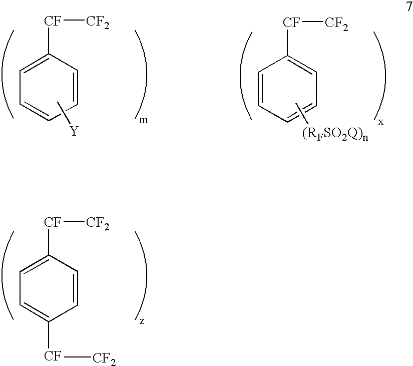
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

Q=OM, OH, NHSO2R_{F}, wherein M=Li⁺, Na⁺, K⁺ or Cs⁺,

n is 1 or 2,

m, x and z are mole fractions wherein m is 0.01 to 0.99

x is 0.99 to 0.01,

z is 0.0001 to 0.10, and

m+x+z=1

In a fifth aspect, the invention provides a membrane electrode assembly comprising a polymer electrolyte membrane, having a first surface and a second surface, wherein the membrane is prepared from a homopolymer or copolymer selected from the group consisting of:

(a) a homopolymer having the structure: 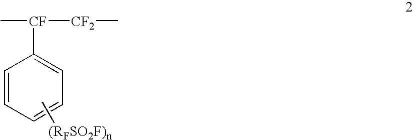
wherein R_{F} is linear or branched perfluoroalkene group, optionally containing oxygen or chlorine,

n is 1 or 2;

(b) a copolymer having the structure: 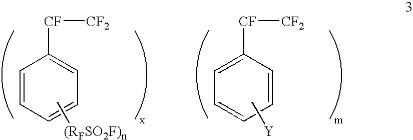
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

n is 1 or 2,

m and x are mole fractions wherein m is 0.01 to 0.99,

x is 0.99 to 0.01; and

x+m=1; and

(c) a copolymer having the structure: 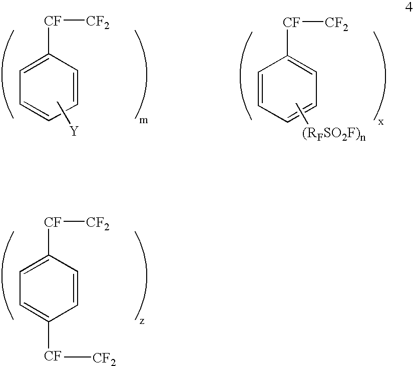
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

n is 1 or 2,

m, x and z are mole fractions wherein m is 0.01 to 0.99,

x is 0.99 to 0.01, and

z is 0.0001 to 0.10

m+x+z=1; and mixtures thereof.

In the fifth aspect, the membrane electrode assembly comprises a polymer electrolyte membrane further comprising a porous support.

In the fifth aspect, the membrane electrode assembly further comprises at least one electrode prepared from an electrocatalyst coating composition present on the first and second surfaces of the membrane. It also further comprises at least one gas diffusion backing. Alternately, the membrane electrode assembly further comprises a gas diffusion electrode present on the first and second surfaces of the membrane, wherein the gas diffusion electrode comprises a gas diffusion backing and an electrode prepared from an electrocatalyst containing composition.

In a sixth aspect, the invention provides an electrochemical cell, such as a fuel cell, comprising a membrane electrode assembly, wherein the membrane electrode assembly comprises a polymer electrolyte membrane, having a first surface and a second surface, wherein the membrane is prepared from a homopolymer or copolymer selected from the group consisting of:

(a) a homopolymer having the structure: 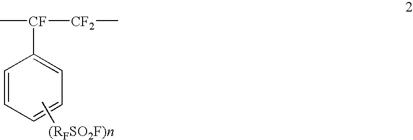
wherein R_{F} is linear or branched perfluoroalkene group, optionally containing oxygen or chlorine,

n is 1 or 2;

(b) a copolymer having the structure: 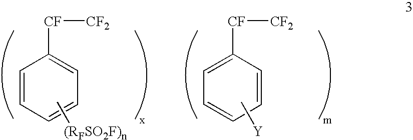
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

n is 1 or 2,

m and x are mole fractions wherein m is 0.01 to 0.99,

x is 0.99 to 0.01,

x+m=1, and

(c) a copolymer having the structure: 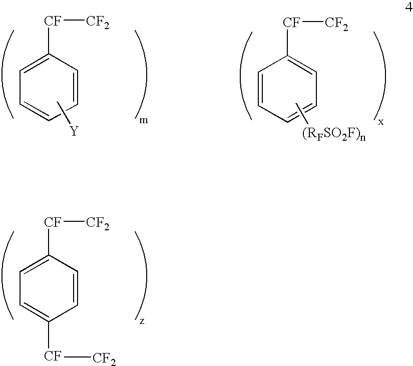
wherein R_{F} is linear or branched perfluoroalkene group, optionally containing oxygen or chlorine,

Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,

n is 1 or 2,

m, x and z are mole fractions wherein m is 0.01 to 0.99,

x is 0.99 to 0.01,

z is 0.0001 to 0.10, and

m+x+z=1; and mixtures thereof.

In the sixth aspect, the invention provides a fuel cell comprising a polymer electrolyte membrane further comprising a porous support.

In the sixth aspect, the fuel cell further comprises at least one electrode prepared from an electrocatalyst containing composition, e.g., an anode and a cathode, present on the first and second surfaces of the polymer electrolyte membrane. It also further comprises at least one gas diffusion backing. Alternately, the membrane electrode assembly further comprises a gas diffusion electrode present on the first and second surfaces of the membrane, wherein the gas diffusion electrode comprises a gas diffusion backing and an electrode prepared from an electrocatalyst containing composition.

In the sixth aspect, the fuel cell further comprises a means for delivering a fuel to the anode, a means for delivering oxygen to the cathode, a means for connecting the anode and cathode to an external electrical load, hydrogen or methanol in the liquid or gaseous state in contact with the anode, and oxygen in contact with the cathode. The fuel is in the liquid or vapor phase. Some suitable fuels include hydrogen, alcohols such as methanol and ethanol; ethers such as diethyl ether, etc.

### BRIEF DESCRIPTION OF DRAWINGS

FIG. 1 is a schematic illustration of a single cell assembly.

FIG. 2 is a schematic illustration of the lower fixture of a four-electrode cell for in-plane conductivity measurement.

### DETAILED DESCRIPTION OF THE INVENTION

The monomers of the invention that are small molecules may be used to prepare homopolymers or copolymers that are useful as electrolytes in the preparation of the solid polymer electrolyte membranes. These polymer electrolyte membranes are used to make catalyst-coated membranes that are a component of fuel cells. These homopolymers or copolymers are also useful as electrolytes in other electrochemical cells, such as batteries, chloralkali cells, electrolysis cells, sensors, electrochemical capacitors, and modified electrodes.

Monomer:

The monomers of the invention have the following structure: 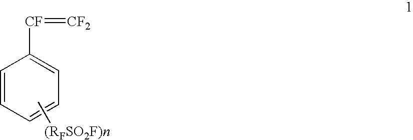
wherein R_{F} is linear or branched perfluoroalkene group, optionally containing oxygen or chlorine, such as (CF₂)ᵣ wherein r=1 to 20, (CF₂CF₂)ᵣOCF₂CF₂ wherein r=0 to 6, CF(CF₃)O)ᵣCF₂CF₂ wherein r=1 to 8, and more typically (CF₂)ᵣ wherein r=1 to 8, (CF₂CF₂)ᵣOCF₂CF₂ wherein r=0 to 2, CF(CF₃)O)ᵣCF₂CF₂ wherein r=1 to 2; and

n is 1 or 2, more typically n is 1.

A. Synthesis of Monomers

Monomer having structure 1 was prepared by the Pd catalyzed reaction of a trifluorovinyl zinc reagent with aryl bromide, which was disclosed in Feiring, et al, J. Fluorine Chem. 105, 129, 2000. The vinyl zinc reagent was made from the reaction of CF₂═CFBr and zinc powder in DMF (Burton et al, JOC 53, 2714, 1988). 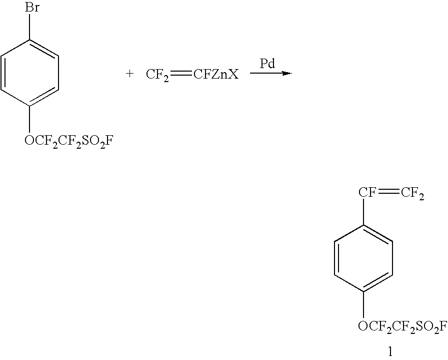
Other monomers such as trifluorostyrene and 1,4-bis(trifluorostyrene) were made in similar fashion according to Burton's method. Alternatively, the monomers can be prepared by reaction of iodohalobenzenes with IR_{F}SO₂F in the presence of copper powder to give the coupling products XC₆H₄R_{F}SO₂F, followed by a palladium catalyzed coupling reaction with CF₂═CFZnX. A third method includes first adding CF₂ClCF₂ICl to iodo- or bromobenzenes to give CF₂ClCFClC₆H₄X wherein X=I, Br, and then coupling with IR_{F}SO₂F in the presence of copper powder to produce the coupled product, CF₂ClCFClC₆H₄R_{f}SO₂F, which was treated with Zn to produce the desired monomer CF₂═CFC₆H₄R_{F}SO₂F. CF₂ClCF₂ICl also reacts with diiodo- or dibromobenzenes to give CF₂ClCFClC₆H₃X₂ wherein X=Br, I, which may be coupled with IRFSO2F and copper powder resulting in CF₂CICFClC₆H₃(R_{F}SO₂F)₂. Finally, dechlorination of CF₂ClCFClC₆H₃(R_{F}SO₂F)₂ with zinc gives CF₂═CFC₆H₃(R_{F}SO₂F)₂.
Homopolymers and Copolymers:

These monomers are used to prepare homopolymers and copolymers using the following procedure:

Homo- and copolymerization of I may be conducted by neat polymerization, solution polymerization, suspension polymerization, or emulsion polymerization. Typical initiators such as Lupersol® 11 and perfluoroacyl peroxide were used in suspension polymerization or solution polymerization. In an aqueous polymerization, inorganic peroxides such as potassium persulfates (KPS) and ammonium persulfate (APS) obtained from Aldrich, Milwaukee, Wis.) were used as an initiator, or fluorinated organic salts such as ammonium perfluorooctanoate and fluorinated alkane sulfonates, or non-fluorinated surfactants such as dodecylaminie hydrochloride salt were used as surfactants. Monomers represented by structure 1 were typically used in aqueous emulsion polymerization. Molecular weight of polymers can be controlled by addition of chain transfer agents such as halocarbons, CHCl₃, fluorinated iodides and bromides, MeOH, ethers esters and alkanes. Polymers were isolated by coagulation. The polymers have high thermal stability and may be pressed into thin films. The polymer also dissolved in certain solvents such as trifluorotoluene and 2,5-dichlorotrifluorotoluene. Thin films may also be cast from these polymer solutions. Slightly crosslinked polymers such as those having the structure 4 have improved mechanical properties and reduced excess water uptake.

The resulting homopolymer formed by the above procedure has the following structure: 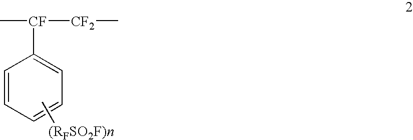
wherein R_{F} is linear or branched perfluoroalkene group, optionally containing oxygen or chlorine, such as (CF₂)ᵣ wherein r=1 to 20, (CF₂CF₂)ᵣOCF₂CF₂ wherein r=0 to 6, CF(CF₃)O)ᵣCF₂CF₂ wherein r=1 to 8, and more typically (CF₂)ᵣ wherein r=1 to 8, (CF₂CF₂)ᵣOCF₂CF₂ wherein r=0 to 2, CF(CF₃)O)ᵣCF₂CF₂ wherein r=1 to 2;

n is 1 or 2, more typically n is 1; and

The resulting copolymer formed using the above procedure are represented by the structure: 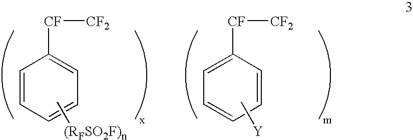
wherein R_{F} is linear or branched perfluoroalkene group, optionally containing oxygen or chlorine, such as (CF₂)ᵣ wherein r=1 to 20, (CF₂CF₂)ᵣOCF₂CF₂ wherein r=0 to 6, CF(CF₃)O)ᵣCF₂CF₂ wherein r=1 to 8, and more typically (CF₂)ᵣ wherein r=1 to 8, (CF₂CF₂)ᵣOCF₂CF₂ wherein r=0 to 2, CF(CF₃)O)ᵣCF₂CF₂ wherein r=1 to 2;

Y is H; halogen such as F, Cl, Br, or I; linear or branched perfluoroalkyl and non-fluorinated alkyl groups, wherein the alkyl group comprises C1 to C10 carbon atoms, such as CₙF₂ₙ₊₁ and CₙH₂ₙ₊₁ wherein n is 1 to 10, more typically C_{q}F_{2q+1} wherein q is 1 to 6; or a perfluoroalkyl group containing oxygen, chlorine or bromine, and wherein the alkyl group comprises C1 to C10 carbon atoms, such as CF₃(CF₂)_{q}O(CF₂CF₂)_{q} wherein q=1 to 5, CF₃CF₂CF₂(OCFCF₃)_{q} wherein q=1 to 5; and more typically CF₃(CF₂)_{q}OCF₂CF₂ wherein q=1 to 2, CF₃CF₂CF₂(OCFCF₃)_{q} wherein q=1 to 3;

n is 1 or 2, more typically 1;

m and x are mole fractions wherein m is 0 to 0.99, more typically 0.1 to 0.4;

x is 1 to 0.001; more typically 0.9 to 0.6; and

x+m=1; and 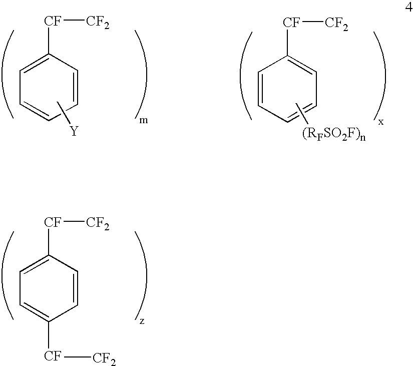
wherein R_{F} is linear or branched perfluoroalkene group, optionally containing oxygen or chlorine, such as (CF₂)ᵣ wherein r=1 to 20, (CF₂CF₂)ᵣOCF₂CF₂ wherein r=0 to 6, CF(CF₃)O)ᵣCF₂CF₂ wherein r=1 to 8, and more typically (CF₂)ᵣ wherein r=1 to 8, (CF₂CF₂)ᵣOCF₂CF₂ wherein r=0 to 2, CF(CF₃)O)ᵣCF₂CF₂ wherein r=1 to 2; and

Y is H; halogen such as F, Cl, Br, or I; linear or branched perfluoroalkyl and non-fluorinated alkyl groups, wherein the alkyl group comprises C1 to C10 carbon atoms, such as CₙF₂ₙ₊₁ and CₙH₂ₙ₊₁, wherein n is 1 to 10, more typically C_{q}F_{2q+1} wherein q is 1 to 6; or a perfluoroalkyl group containing oxygen, chlorine or bromine, and wherein the alkyl group comprises C1 to C10 carbon atoms, such as CF₃(CF₂)_{q}O(CF₂CF₂)_{q} wherein q=1 to 5, CF₃CF₂CF₂(OCFCF₃)_{q} wherein q=1 to 5; and more typically CF₃(CF₂)_{q}OCF₂CF₂ wherein q=1 to 2, CF₃CF₂CF₂(OCFCF₃)_{q} wherein q=1 to 3;

n is 1 or 2, more typically 1;

m, x and z are mole fractions wherein m is 0.1 to 0.8, more typically 0.2 to 0.6;

x is 0.2 to 0.9, more typically 0.4 to 0.8; and

z is 0.001 to 0.05, more typically 0.002 to 0.01.

Membrane:

The hompolymer and copolymers can be cast into thin films from their solutions. Typically, THF, trifluorotoluene were used as solvents. The cast films were transparent and flexible. The films also may also be made by thermally pressing at 200 to 250° C. The films may be hydrolyzed with bases such as MOH, M₂CO₃ wherein M=Li⁺, Na⁺, K⁺ or Cs⁺, or MOH in a mixture of MeOH, DMSO and water. The hydrolysis is typically carried out at room temperature to 373° F., preferably at room temperature to 323° F. Treatment of polymeric salts with acids such as HNO₃ gave polymeric acids. Polymers represented by structures 2, 3 and 4 may be converted to the corresponding sulfonimide by reaction with CF₃SO₂NH₂ and base.

The ionomers of homopolymers and copolymers identified above may be imbibed into a porous support to form a polymer electrolyte membrane having improved mechanical properties and dimensional stability. These membranes are capable of operating at a temperature of above 100° C. Ionomers may have 5% to 99.9% of membrane weight, typically, 20 to 98%, more typically 50 to 90%.

Porous Support:

The porous support of the membrane may be made from a wide range of components. The porous support of the present invention may be made from a hydrocarbon such as a polyolefin, e.g., polyethylene, polypropylene, polybutylene, copolymers of those materials, and the like. Perhalogenated polymers such as polychlorotrifluoroethylene may also be used. For resistance to thermal and chemical degradation, the support preferably is made of a highly fluorinated polymer, most preferably perfluorinated polymer.

For example, the polymer for the porous support can be a microporous film of polytetrafluoroethylene (PTFE) or a copolymer of tetrafluoroethylene with other perfluoroalkyl olefins or with perfluorovinyl ethers. Microporous PTFE films and sheeting are known which are suitable for use as a support layer. For example, U.S. Pat. No. 3,664,915 discloses uniaxially stretched film having at least 40% voids. U.S. Pat. Nos. 3,953,566, 3,962,153 and 4,187,390 disclose porous PTFE films having at least 70% voids.

Alternatively, the porous support may be a fabric made from fibers of the support polymers discussed above woven using various weaves such as the plain weave, basket weave, leno weave, or others. A membrane suitable for the practice of the invention can be made by coating the porous support fabric with the compound of the invention to form a composite membrane. To be effective the coating must be on both the outside surfaces as well as distributed through the internal pores of the support. This may be accomplished by impregnating the porous support with a solution or dispersion of the polymer suitable for the practice of the invention using a solvent that is not harmful to the polymer or the support, and under impregnation conditions that can form a thin, even coating of the polymer on the support. The support with the solution/dispersion is dried to form the membrane. If desired, thin films of the ion exchange polymer can be laminated to one or both sides of the impregnated porous support to prevent bulk flow through the membrane that can occur if large pores remain in the membrane after impregnation.

It is preferred for the compound to be present as a continuous phase within the membrane.

Other forms of the solid polymer electrolyte membrane include the PTFE yarn embedded type and the PTFE fibril dispersed type, wherein the PTFE fibril is dispersed in the ion exchange resin as disclosed in 2000 Fuel Cell Seminar (10/30 to 11/2, 2000, Portland, Oreg.) Abstracts, p-23.

Electrochemical Cell:

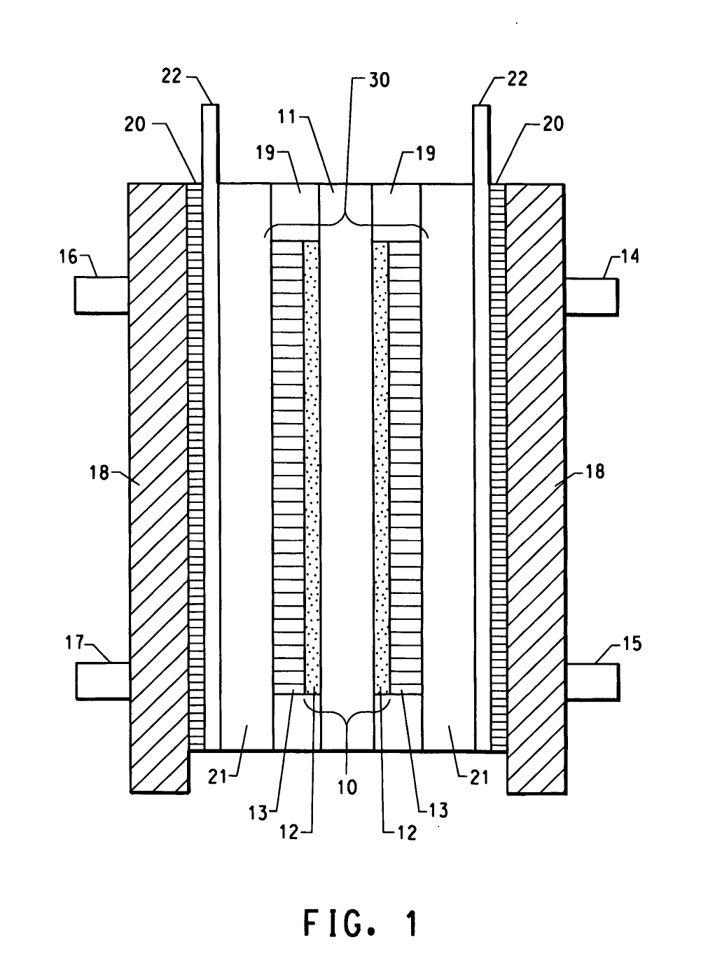

As shown in FIG. 1, an electrochemical cell, such as a fuel cell, comprises a catalyst-coated membrane (CCM) (10) in combination with at least one gas diffusion backing (GDB) (13) to form an unconsolidated membrane electrode assembly (MEA). The catalyst-coated membrane (10) comprises an ion exchange polymer membrane (11) discussed above and catalyst layers or electrodes (12) formed from an electrocatalyst coating composition. The fuel cell is further provided with an inlet (14) for fuel, such as liquid or gaseous alcohols, e.g. methanol and ethanol; or ethers such as diethyl ether, etc., an anode outlet (15) a cathode gas inlet (16) a cathode gas outlet (17) aluminum end blocks (18) tied together with tie rods (not shown), a gasket for sealing (19) an electrically insulating layer (20) and graphite current collector blocks with flow fields for gas distribution (21) and gold plated current collectors (22).

The fuel cell utilizes a fuel source that may be in the liquid or gaseous phase, and may comprise hydrogen, an alcohol or ether. Typically a methanol/water solution is supplied to the anode compartment and air or oxygen supplied to the cathode compartment.

Catalyst Coated Membrane (CCM):

A variety of techniques are known for CCM manufacture which apply an electrocatalyst coating composition similar to that described above onto the solid fluorinated polymer electrolyte membrane. Some known methods include spraying, painting, patch coating and screen, decal, pad or flexographic printing.

In one embodiment of the invention, the MEA (30), shown in FIG. 1, may be prepared by thermally consolidating the gas diffusion backing (GDB) with a CCM at a temperature of under 200° C., preferably 140-160° C. The CCM may be made of any type known in the art. In this embodiment, an MEA comprises a polymer electrolyte (SPE) membrane with a thin catalyst-binder layer disposed thereon. The catalyst may be supported (typically on carbon) or unsupported. In one method of preparation, a catalyst film is prepared as a decal by spreading the catalyst ink on a flat release substrate such as Kapton® polyimide film (available from the DuPont Company). After the ink dries, the decal is transferred to the surface of the SPE membrane by the application of pressure and heat, followed by removal of the release substrate to form a catalyst coated membrane (CCM) with a catalyst layer having a controlled thickness and catalyst distribution. Alternatively, the catalyst layer is applied directly to the membrane, such as by printing, and then the catalyst film is dried at a temperature not greater than 200° C.

Membrane Electrode Assembly:

The CCM, thus formed, is then combined with a GDB to form the MEA (30). The MEA is formed, by layering the CCM and the GDB, followed by consolidating the entire structure in a single step by heating to a temperature no greater than 200° C., preferably in the range of 140-160° C., and applying pressure. Both sides of the MEA can be formed in the same manner and simultaneously. Also, the composition of the catalyst layer and GDB could be different on opposite sides of the membrane. Alternately, the membrane electrode may be formed by placing gas diffusion electrode each surface of the polymer electrolyte membrane, wherein the gas diffusion electrode comprises a gas diffusion backing and an electrode prepared from an electrocatalyst containing composition. The electrocatalyst composition may comprise the homopolymers or copolymers of the invention as a binder in the composition.

The invention is illustrated in the following examples.

### EXAMPLES

Liquid Conductivity Measurement

The conductivity of a liquid is measured using a cell capable of handling corrosive samples at elevated temperature with volumes as small as 800 μL. Two coil electrodes are formed 25 mm apart by wrapping 0.38 mm diameter platinum wires five times around one end of a 5.14 mm diameter Macor® machinable glass ceramic rod (Corning Inc., Corning, N.Y.) and insulating the remainder of the wire leads with heat-shrink PTFE tubing. The sample is loaded into a 9 mm outside diameter×6.8 mm inside diameter×178 mm length glass tube and the rod is inserted so that the electrodes are completely immersed in the sample. The tube is placed into a forced-convection thermostated oven for heating. The real part of the AC impedance, Rₛ, is measured at a frequency of 1 kHz using a potentiostat/frequency response analyzer (PC4/750™ with EIS software, Gamry Instruments, Warminster, Pa.). The phase angles are typically less than 2 degrees, which indicates that the measurement is unaffected by capacitive contributions from the electrode interfaces. The cell constant, K, is determined by measuring the real part of the impedance, R_{c}, at a frequency of 10 kHz using a NIST traceable potassium chloride conductivity calibration standard for nominal 0.1 S/cm (0.1027 S/cm actual) and calculating as
K=R_{c}×0.1027 S/cm×(1+ΔT×0.02° C.⁻¹)
where ΔT is the difference between the temperature of the calibration standard, Tₘ, and 25° C. (ΔT=Tₘ−25). The cell constant is typically close to 12 cm⁻¹. The conductivity, K, of the sample is then calculated as
κ=K/Rₛ
Through-Plane Conductivity Measurement

The through-plane conductivity of a membrane is measured by a technique in which the current flows perpendicular to the plane of the membrane. The lower electrode is formed from a 12.7 mm diameter stainless steel rod and the upper electrode is formed from a 6.35 mm diameter stainless steel rod. The rods are cut to length, and their ends are polished and plated with gold. A stack is formed consisting of lower electrode/GDE/membrane/GDE/upper electrode. The GDE (gas diffusion electrode) is a catalyzed ELAT® (E-TEK Division, De Nora North America, Inc., Somerset, N.J.) comprising a carbon cloth with microporous layer, platinum catalyst, and 0.6-0.8 mg/cm² Nafion® application over the catalyst layer. The lower GDE is punched out as a 9.5 mm diameter disk, while the membrane and the upper GDE are punched out as 6.35 mm diameter disks to match the upper electrode. The stack is assembled and held in place within a block of Macor® machinable glass ceramic (Corning Inc., Corning, N.Y.) that has a 12.7 mm diameter hole drilled into the bottom of the block to accept the lower electrode and a concentric 6.4 mm diameter hole drilled into the top of the block to accept the upper electrode. A force of 270 N is applied to the stack by means of a clamp and calibrated spring. This produces a pressure of 8.6 MPa in the active area under the upper electrode, which insures a low impedance ionic contact between the GDE's and the membrane. The fixture is placed in a forced-convection thermostated oven for heating. The real part of the AC impedance of the fixture containing the membrane, Rₛ, is measured at a frequency of 100 kHz using a potentiostat/frequency response analyzer (PC4/750™ with EIS software, Gamry Instruments, Warminster, Pa.). The fixture short, R_{f}, is also determined by measuring the real part of the AC impedance at 100 kHz for the fixture and stack assembled without a membrane sample. The conductivity, K, of the membrane is then calculated as
κ=t/((Rₛ−R_{f})×0.317 cm²),
where t is the thickness of the membrane in cm.
In-Plane Conductivity Measurement

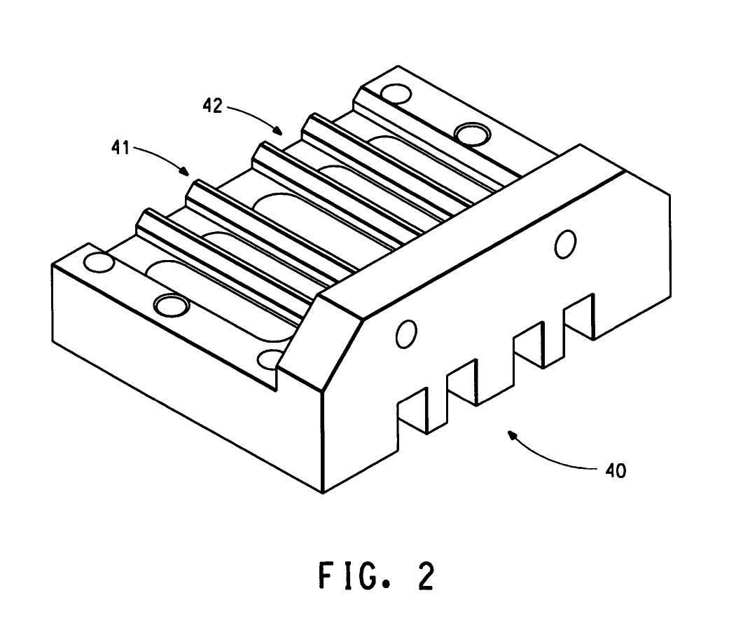

The in-plane conductivity of a membrane is measured under conditions of controlled relative humidity and temperature by a technique in which the current flows parallel to the plane of the membrane. A four-electrode technique is used similar to that described in an article entitled “Proton Conductivity of Nafion® 117 As Measured by a Four-Electrode AC Impedance Method” by Y. Sone et al., J. Electrochem. Soc., 143, 1254 (1996), which is herein incorporated by reference. Referring to FIG. 2, a lower fixture (40) is machined from annealed glass-fiber reinforced PEEK to have four parallel ridges (41) containing grooves that support and hold four 0.25 mm diameter platinum wire electrodes. The distance between the two outer electrodes is 25 mm, while the distance between the two inner electrodes is 10 mm. A strip of membrane is cut to a width between 10 and 15 mm and a length sufficient to cover and extend slightly beyond the outer electrodes, and placed on top of the platinum electrodes. An upper fixture (not shown), which has ridges corresponding in position to those of the bottom fixture, is placed on top and the two fixtures are clamped together so as to push the membrane into contact with the platinum electrodes. The fixture containing the membrane is placed in a small pressure vessel (pressure filter housing), which is placed in a forced-convection thermostated oven for heating. The temperature within the vessel is measured by means of a thermocouple. Water is fed from a calibrated Waters 515 HPLC pump (Waters Corporation, Milford, Mass.) and combined with dry air fed from a calibrated mass flow controller (200 sccm maximum) to evaporate the water within a coil of 1.6 mm diameter stainless steel tubing inside the oven. The resulting humidified air is fed into the inlet of the pressure vessel. The total pressure within the vessel (100 to 345 kPa) is adjusted by means of a pressure-control letdown valve on the outlet and measured using a capacitance manometer (Model 280E, Setra Systems, Inc., Boxborough, Mass.). The relative humidity is calculated assuming ideal gas behavior using tables of the vapor pressure of liquid water as a function of temperature, the gas composition from the two flow rates, the vessel temperature, and the total pressure. Referring to FIG. 2, the slots (42) in the lower and upper parts of the fixture allow access of humidified air to the membrane for rapid equilibration with water vapor. Current is applied between the outer two electrodes while the resultant voltage is measured between the inner two electrodes. The real part of the AC impedance (resistance) between the inner two electrodes, R, is measured at a frequency of 1 kHz using a potentiostat/frequency response analyzer (PC4/750™ with EIS software, Gamry Instruments, Warminster, Pa.). The conductivity, κ, of the membrane is then calculated as
κ=1.00 cm/(R×t×w),
where t is the thickness of the membrane and w is its width (both in cm).

### Example 1

p-CF₂═CFC₆H₄OCF₂CF₂SO₂F was prepared using the following procedure:

A 1000 mL two necked flask fitted with rubber septa, a magnetic stirring bar, vented connector tube and a dry ice condenser vented to a nitrogen purge tube bubbler was charged with 45 g (0.69 mol) of acid-washed Zn and 500-mL of DMF at room temperature. CF₂═CFBr was slowly added as a gas via the vented connector tube and allowed to condense at the dry ice to a suspension of Zn and DMF mixture in the flask After addition of 2 mL of bromine, an exothermic reaction occurred and the mixture was stirred at room temperature for 2 hrs, during which 99.1 g (0.616 mol) of CF₂═CFBr were added, and then stirred at 65° C. for 1.5 hrs to give CF₂═CFZnX solution.

A 1 L three necked flask fitted with a magnetic stirring bar, and a cold water condenser was charged with 6.0 g of Pd(PPh₃)₄, and then 560 mL of (CF₂═CF)ZnX solution prepared as described above was transferred via a cannula to the reaction flask. 115 g (0.324 mol) of p-Br—(C₆H₄)OCF₂CF₂SO₂F were added via a syringe and the reaction mixture was heated to 75° C. for 10 hours. The condenser was replaced with a distillation head and distilled at 0.30-1.8 mmHg to a receiver that was cooled with dry ice. About 500 mL of a clear light yellow liquid was obtained. This clear light yellow liquid was poured into water and the lower layer separated, washed with two times its volume of ice cold water, and then with a brine solution to recover 101.56 g. of crude product. Distillation gave 99 g (86%) of pure product, bp 57-61° C./0.5 mmHg. ¹⁹F NMR: +43.8 (m, 1F), −82.0 (s, 2F), −99.7(dd, J=67.8 Hz, J=34 Hz, 1F), −112.0 (s, 2F), −114.4 (dd, J=109.3 Hz, J=67.8 Hz, 1F), −177.4 (dd, J=109.3 Hz, J=34 Hz, 1F). ¹H NMR: 7.6 (d, J=8 Hz, 2H), 7.2 (d, J=8 Hz, 2H).

### Example 2

CF₂═CFC₆H₄OCF₂CF₂SO₂F was polymerized using ammonium persulfate (APS):

A 250 mL three necked round bottomed flask, fitted with a rubber septa, cold-water condenser with a N₂ outlet/inlet and bubbler, a magnetic stirring bar, and thermocouple, was charged with 50 mL of deionized water and 4.8 mL 20 wt. % C8 solution (aq). The solution was bubbled with N₂ for 30 min. then 7.2 g (20.2 mmol) of CF₂═CFC₆H₄OCF₂CF₂SO₂F were added to the flask via a syringe under N₂, and then ultrasonicated for min. After heating to 50° C., 50 mg of APS in 2 mL of water were added and the resulting mixture was stirred at 50° C. over weekend, and then an additional 10 mg of APS in 1 mL of water were added. Stirring of the mixture was continued for 14 hrs and then the mixture was frozen. After melting, the mixture was treated with 15 ml of 10% HNO₃ at 90° C. for 1.5 hrs, then cooled to RT. The resulting solids were filtered and washed with water three times and further dried at 105° C. under vacuum/N₂ for 4 hrs to give 5.41 of polymer, which was soluble in THF. ¹H NMR(THF-d8): 6.0-7.8 ppm. ¹⁹F NMR: +40.2 (s, 1F), −84.6 (m, 2F), −107 to −133 (m, 2F), −114.8 (s, 2F), −175 to −178 (m, 1F). Analysis: calcd for C₁₀H₄F₈SO₃: C, 33.71; H, 1.13; F, 42.70; S, 8.98. Found: C, 33.84; H, 0.93; F, 42.7; S, 8.98. Mw=9.39×10⁵, Mn=1.05×10⁵. DSC indicated the polymer had a Tg of 165° C. TGA indicated decomposition temperature was 300° C. and 10% weight loss was 340° C. in air when heated at 10° C./min.

### Example 3

CF₂═CFC₆H₄OCF₂CF₂SO₂F was polymerized using the following procedure:

A 500 mL three necked round bottomed flask, fitted with a rubber septa, cold water condenser with a N₂ outlet/inlet and bubbler; a magnetic stirring bar and thermocouple, was charged with 100 mL of deionized water and 8.0 mL of 20 wt. % C8 solution (aq). The solution was bubbled with N₂ for 30 min. then 14.4 g (40.4 mmol) of CF₂═CFC₆H₄OCF₂CF₂SO₂F were added to the flask via a syringe under N₂ and the mixture ultrasonicated for 5 min. After heating to 55° C., 57 mg of KPS in 2 mL of water were added and the resulting mixture was stirred at 55° C. for 26 hrs. An additional 28 mg of KPS in 2 mL of water were then added. Stirring of the mixture was continued for 22 hrs and the mixture was frozen. After melting, the mixture was treated with 50 ml of 10% HNO₃ at 90° C. for 1.5 hrs, then cooled to RT. The resulting solids were filtered and washed with DI water three times to give a fine off-white polymer with some larger solids. The solids were slurried in methanol, filtered and air-dried. After air-drying overnight, 15.84 g of a white solid were recovered. The sample was further dried at 100° C. under vacuum/N₂ overnight and 12.0 g. of sample having Mw=8.44×10⁵ and Mn=2.02×10⁵ were recovered.

### Example 4

CF₂═CFC₆H₄OCF₂CF₂SO₂F was polymerized using APS at 80° C. and the following procedure:

A 100 mL three necked round bottomed flask, fitted with a rubber septa, cold water condenser with a N₂ outlet/inlet and bubbler; a magnetic stirring bar and thermocouple, was charged with 40 mL of deionized water and 3.8 mL 20 wt. % C8 solution (aq). The solution was bubbled with N₂ for 75 min and then 5.6 g (15.7 mmol) of F₂═CFC₆H₄OCF₂CF₂SO₂F were added to the flask via a syringe under N₂. After heating to 80° C., 4 mg of APS in 1 mL of water were added to the flask with stirring every hour for 11 hours. The mixture was then frozen in the flask. After melting, the mixture was treated with 10 ml of 10% HNO₃ at 90° C. for 1.5 hrs, and then cooled to RT. The resulting solids were filtered and washed with water three times and further dried at 105° C. under vacuum/N₂ for 5 hrs to give 4.98 g of polymer (88.9%) having a Mw=4.44×10⁵ and a Mn=7.16×10⁴

### Example 5

CF₂═CFC₆H₄OCF₂CF₂SO₂F was prepared in the presence of CHCl₃ using APS CHCl₃ at 80° C., and the following procedure:

A 100 mL three necked round bottomed flask, fitted with rubber septa, cold water condenser with a N₂ outlet/inlet and bubbler; a magnetic stirring bar and thermocouple, was charged with 40 mL of deionized water and 3.8 mL 20 wt. % C8 aqueous solution. The solution was bubbled with N₂ for 75 min., followed by the addition of 5.6 g (15.7 mmol) of CF₂═CFC₆H₄OCF₂CF₂SO₂F and 10 μl of CHCl₃ via a syringe under N₂. After heating to 80° C., 4 mg of APS in 1 mL of water were added to the flask with stirring every hour for 11 hours. The mixture was then frozen. After melting, the mixture was treated with 10 ml of 10% HNO₃ at 90° C. for 1.5 hrs, and then cooled to RT. The resulting solids were filtered and washed with water three times and further dried at 110° C. under vacuum/N₂ for 5 hrs to give 4.46 g of polymer (79.6%) having a Mw=2.04×10⁵ and an Mn=5.62×10⁴

### Example 6

p-CF₂═CFC₆H₄OCF₂CF₂SO₂F polymer was prepared in the presence of I(CF₂)₆I using APS at 80° C., and the following procedure:

A 100 mL three necked round bottomed flask, fitted with a rubber septa, cold water condenser with a N₂ outlet/inlet and bubbler; a magnetic stirring bar and thermocouple, was charged with 40 mL of deionized water and 3.8 mL 20 wt. % C8 solution (aq). The solution was bubbled with N₂ for 75 min, and then 5.6 g (15.7 mmol) of CF₂═CFC₆H₄OCF₂CF₂SO₂F and 35 mg of I(CF2)₆I were added to the flask via a syringe under N₂. After heating to 80° C., 4 mg of APS in 1 mL of water were added to the flask with stirring every hour for 11 hours. The mixture was then frozen. After melting, the mixture was treated with 10 ml of 10% HNO₃ at 90° C. for 1.5 hrs, and then cooled to RT. The resulting solids were filtered and washed with water three times and further dried at 105° C. under vacuum/N₂ for 5 hrs to give 4.18 g of (79.1%) of polymer having a Mw=5.39×10⁵ and Mn=1.10×10⁴

### Example 7

p-CF₂═CFC₆H₄OCF₂CF₂SO₂F was copolymerized with trifluorostyrene using the following procedure:

A three-necked clean flask, fitted with an N₂ inlet/outlet, a magnetic stirring bar and a thermocouple, was charged with 25 mL of deionized water and 2.1 mL of 20% C8 aqueous solution. The solution was bubbled with N₂ for 30 min. 2.9 g of CF₂═CFC₆H₄OCF₂CF₂SO₂F and 1.3 g (8.2 mmol) of CF₂═CFC₆H₅ were added to the flask via a syringe under N₂ purge. After heating to 55° C. (temperature control is very important!), 52 mg of Potassium persulfate (KPS) in 2 mL of deionized water were added via a syringe and the flask was kept at 55° C. for 24 hrs. An additional 15 mg of KPS were added and stirred for another 24 hrs. The flask was cooled with dry ice to freeze, and then warmed up to RT. After 15 ml of 10% HNO₃ were added, the mixture was stirred at 90° C. for 1.5 hrs, cooled to RT, filtered and washed with water 3 times to give white powder with some beige chunks. This was further dried in a vacuum oven at 100° C. for 4 hrs to give 2.81 g of copolymer.

### Examples 8

p-CF₂═CFC₆H₄OCF₂CF₂SO₂F was copolymerized with C₆H₅CF═CF₂ using the following procedure:

A three-necked 100 mL flask, fitted with an N₂ inlet/outlet, a magnetic stirring bar and a thermocouple, was charged with 40 mL of deionized water and 3.8 mL of 20% ammonium perfluorooctanoate (C8) aqueous solution. The solution was bubbled with N₂ for 30 min. 5.0 g (14 mmol) g of CF₂═CFC₆H₄OCF₂CF₂SO₂F and 0.63 g (4 mmol) of CF₂═CFC₆H₅ were added to the flask via a syringe under N₂ purge and ultrasonicated for 5 min. After heating to 55° C., 52 mg of potassium persulfate (KPS) in 2 mL of deionized water were added via a syringe and the flask was kept at 55° C. for 24 hrs. An additional 32 mg of KPS were added and the mixture was stirred over the weekend. The flask was cooled with dry ice to freeze, and then warmed up to RT. After 20 ml of 10% HNO₃ were added, the mixture was stirred at 90° C. for 1.5 hrs, then cooled to RT. The solids were slurried in 100 mL DI water three times to neutral of pH 6. 7.19 g of a white solid were recovered after air-drying overnight. The sample was further dried at 120° C. under vacuum/N₂ overnight, recovering 4.00 g. of a beige particulate solid. ¹⁹F NMR in THF solution indicated composition of (CF₂CFC₆H₄OCF₂CF₂SO₂F)_{7.4}(CF₂CFC₆H₅), Mw=1.42×10⁵, Mn=7.84×10³

### Example 9

CF₂═CFC₆H₄OCF₂CF₂SO₂F was copolymerized with C₆H₅CF═CF₂@ 1:3 mole ratio using the following procedure:

A 250 mL three necked round bottomed flask, fitted with rubber septa, cold water condenser with a N₂ outlet/inlet and bubbler; a magnetic stirring bar and thermocouple, was charged with 50 mL of deionized water and 1.0 g of dodecylamine hydrochloride. The solution was bubbled with N₂ for 30 min, and then 2.8 g (7.86 mmol) g of CF₂═CFC₆H₄OCF₂CF₂SO₂F and 3.7 g (23.4 mmol) of C₆H₅CF═CF₂ were added to the flask via a syringe under N₂ and ultrasonicated for 5 min. After heating to 55° C., 57 mg of KPS in 2 mL of water were added and the resulting mixture was stirred for 18 hrs, and then additional 15 mg of KPS were added. After being stirred for 33 hrs, the mixture was frozen with dry ice overnight and warmed to RT. 50 ml of 10% HNO₃ were added, and the mixture was stirred at 90° C. for 1.5 hrs. The resulting solids were too fine to filter and were recovered by centrifuging. The solids were slurried in 200 mL DI water two times and 150 mL 0.5N NaHCO₃ were added two times to result in a pH 6. Recovery was 2.79 g of a white solid after air-drying overnight. Sample was further dried at 100° C. under vacuum/N₂ overnight recovering 2.76 g. of solids. ¹⁹F NMR indicated that composition was (CF₂CFC₆H₄OCF₂CF₂SO₂F)(C₆H₅CFCF₂)_{2.66}+43.2 (m), −82.3 (m), −107.7 to −110.0, −112.5 (m), −170.5 to −179.7 (m) ppm. Mw=3.04×10⁴ and Mn=3.69×10³

### Example 10

p-CF₂═CFC₆H₄CF₂CF₂SO₂F was copolymerized with C₆H₅CF═CF₂ using the following procedure:

A 250 mL three necked round bottomed flask, fitted with a rubber septa, cold water condenser with a N₂ outlet/inlet and bubbler; a magnetic stirring bar and thermocouple, was charged with 50 mL of deionized water and 1.0 g of dodecylamine hydrochloride. The solution was bubbled with N₂ for 30 min. 4.5 g (12.67 mmol) g of CF₂═CFC₆H₄OCF₂CF₂SO₂F and 2.17 g (13.72 mmol) of C₆H₅CF═CF₂ were added to the flask via a syringe under N₂, and then ultrasonicated for 5 min. After heating to 50° C., 52 mg of KPS in 2 mL of water were added and the mixture was stirred for 25 hrs and an additional 15 mg of KPS in 2 mL of water were added. Stirring was continued over the weekend, and the mixture was frozen with dry ice overnight, and warmed to RT. 40 mL of saturated K₂CO₃ solution were added, solids were isolated by centrifugation, washed with water and MeOH and dried in a vacuum oven to give 2.35 g of white polymer. ¹⁹F NMR indicated that composition was (CF₂CFC₆H₄OCF₂CF₂SO₂F)(C₆H₅CFCF₂) and +43.2 (m), −82.3 (m), −107.7 to −110.0, −1 12.5 (m), −170.5 to −179.7 (m). Mw=9.33×10³ and Mn=1.17×10³.

### Example 11

p-CF₂═CFC₆H₄OCF₂CF₂SO₂F and CF₂═CFC₆H₅CF═CF₂ were copolymerized using the following procedure:

A 100 mL three necked round bottomed flask fitted with a rubber septa, cold water condenser with a N₂ outlet/inlet and bubbler; a magnetic stirring bar and thermocouple was charged with 40 mL of deionized water and 0.1 g of poly(difluoromethylene), alpha-fluoro-omega (2-sulfoethyl) ammonium salt (TLF-8927 fluorosurfactant, E. I. DuPont de Nemours and Co., Wilmington, Del.). The solution was bubbled with N₂ for 30 min, followed by the addition of 7.22 g (20.27 mmol) of CF₂═CFC₆H₄OCF₂CF₂SO₂F and 0.158 g (0.66 mmol) of p-CF₂═CF—C₆H₅—CF═CF₂ to the flask via a syringe under N₂. After heating to 55° C., 52 mg of KPS in 2 mL of water were added and the mixture was stirred for 24 hrs. An additional 15 mg of KPS in 2 mL of water were added. After stirring for 63 hrs, the mixture was frozen on dry ice overnight, and warmed to RT. 15 ml of 10% HNO₃ were added, and the mixture was stirred at 90° C. for 1.5 hrs After cooling to RT, the mixture was filtered and washed with water thrice to give a whitish powder, which was dried in a vacuum oven at 100° C. for 4 hrs to give 5.728 g. of a fine beige powder.

### Example 12

CF₂═CF(C₆H₄)OCF₂CF₂SO₂F and p-CF₂═CF—(C₆H₅)—CF═CF₂ were copolymerized using the following procedure:

A 100 mL three necked round bottomed flask fitted with a rubber septa, cold water condenser with a N₂ outlet/inlet and bubbler; a magnetic stirring bar and thermocouple was charged with 40 mL of deionized water and 0.1 g of poly(difluoromethylene), alpha-fluoro-omega (2-sulfoethyl) ammonium salt (TLF-8927 fluorosurfactant, DuPont). The solution was bubbled with N₂ for 30 min., followed by the addition of 7.22 g (20.27 mmol) of CF₂═CFC₆H₄OCF₂CF₂SO₂F and 0.338 g (1.42 mmol) of p-CF₂═CF—C₆H₅—CF═CF₂ to the flask via a syringe under N₂. After heating to 55° C., 52 mg of KPS in 2 mL of water were added, and the mixture was stirred for 24 hrs. An additional 15 mg of KPS in 2 mL of water were added. After continued stirring for 63 hrs, the mixture was frozen on dry ice overnight, and then warmed to RT. 15 ml of 10% HNO₃ were added. The mixture was stirred at 90° C. for 1.5 hrs, cooled to RT, filtered and washed with water thrice to give a whitish powder, that was dried in a vacuum oven at 100° C. for 4 hrs to give 5.718 g. of a fine beige powder.

### Example 13

CF₂═CFC₆H₄OCF₂CF₂SO₂F and p-CF₂═CFC₆H₅CF═CF₂ were copolymerized using the following procedure:

A 100 mL three necked round bottomed flask fitted with a rubber septa, cold water condenser with a N₂ outlet/inlet and bubbler; a magnetic stirring bar and thermocouple was charged with 40 mL of deionized water and 0.1 g of poly(difluoromethylene), alpha-fluoro-omega (2-sulfoethyl) ammonium salt (TLF-8927 fluorosurfactant, DuPont). The solution was bubbled with N₂ for 30 minutes followed by the addition of 7.22 g (20.27 mmol) of CF₂═CFC₆H₄OCF₂CF₂SO₂F and 0.03 g (0.126 mmol) of p-CF₂═CF—C₆H₅—CF═CF₂ to the flask via a syringe under N₂. After heating to 55° C., 52 mg of KPS in 2 mL of water were added and the mixture was stirred for 24 hrs. An additional 15 mg of KPS in 2 mL of water were added. After continued stirring for 90 hrs, the mixture was frozen on dry ice overnight and warmed to RT. 15 ml of 10% HNO₃ were added, the mixture was stirred at 90° C. for 1.5 hrs, cooled to RT, filtered and washed with water thrice to give a whitish powder. This whitish powder was dried in a vacuum oven at 100° C. for 4 hrs to give 5.94 g. of a fine beige powder.

### Example 14

CF₂═CFC₆H₄OCF₂CF₂SO₂F, C₆H₅CF═CF₂ and p-CF₂═CFC₆H₅CF═CF₂ were copolymerized using the following procedure:

A 100 mL three necked round bottomed flask fitted with a rubber septa, cold water condenser with a N₂ outlet/inlet and bubbler; a magnetic stirring bar and thermocouple was charged with 25 mL of deionized water and 0.05 g of poly(difluoromethylene), alpha-fluoro-omega (2-sulfoethyl) ammonium salt (TLF-8927 fluorosurfactant; DuPont). The solution was bubbled with N₂ for 30 min. followed by the addition of 4.1 g (11.5 mmol) of CF₂═CFC₆H₄OCF₂CF₂SO₂F, 1.28 g (8.1 mmol) of C₆H₅CF═CF₂ and 0.07 g (0.29 mmol) of p-CF₂═CF—C₆H₅—CF═CF₂ to the flask via a syringe under N₂. After heating to 55° C., 52 mg of KPS in 2 mL of water were added and the mixture was stirred for 24 hrs and an additional 15 mg of KPS in 2 mL of water were added. After continued stirring for 48 hrs, the mixture was frozen on dry ice overnight and warmed to RT. 15 ml of 10% HNO₃ were added, the mixture was stirred at 90° C. for 1.5 hrs, cooled to RT and centrifuged to give 2.14 g of yellow liquid and solid that was washed with toluene and dried in a vacuum oven at 110° C. to give 1.15 g of white solid.

### Example 15

Copolymers were hydrolyzed using the following procedure:

The copolymer made in Example 7 was pressed into a thin film at 260° C. The film was immersed in 20% KOH in MeOH, water and DMSO in a ratio of 4:5:1 at room temperature for two hrs. After being washed with water thrice, the film was treated with 10% HNO₃ at 40° C. for 6 hrs, at room temperature overnight, and then treated with fresh 10% HNO₃ again for two hrs. After being washed with de-inoized water, the film had conductivity of 240 mS/cm at 80° C. and 95% relative humility (RH) that was measured using the in-plane method.

What is claimed is:

1. A monomer having the following structure: 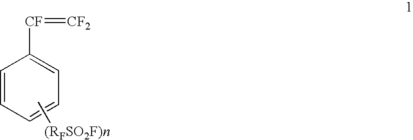
wherein R_{F} is linear or branched perfluoroalkene group, optionally containing oxygen or chlorine; and
n is 1 or 2.

2. The monomer of claim 1 wherein R_{F} is selected from the group consisting of (CF₂)ᵣ wherein r=1 to 20, (CF₂CF₂)ᵣOCF₂CF₂ wherein r=0 to 6, and (CF(CF₃)O)ᵣCF₂CF₂ wherein r=1 to 8.

3. The monomer of claim 2 wherein R_{F} is selected from the group consisting of (CF₂)ᵣ wherein r=1 to 8, (CF₂CF₂)ᵣOCF₂CF₂ wherein r=0 to 2, and (CF(CF₃)O)ᵣCF₂CF₂ wherein r=1 to 2.

4. The monomer of claim 1 wherein n is 1.

5. A homopolymer having the following structure: 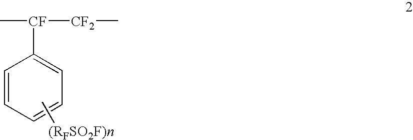
wherein R_{F} is linear or branched perfluoroalkene group, optionally containing oxygen or chlorine,
n is 1 or 2.

6. The homopolymer of claim 5 wherein R_{F} is selected from the group consisting of (CF₂)ᵣ wherein r=1 to 20, (CF₂CF₂)ᵣOCF₂CF₂ wherein r=0 to 6, and (CF(CF₃)O)ᵣCF₂CF₂ wherein r=1 to 8.

7. The homopolymer of claim 6 wherein R_{F} is selected from the group consisting of (CF₂)ᵣ wherein r=1 to 8, (CF₂CF₂)ᵣOCF₂CF₂ wherein r=0 to 2, and (CF(CF₃)O)ᵣCF₂CF₂ wherein r=1 to 2.

8. The homopolymer of claim 1 wherein n is 1.

9. A copolymer selected from the group consisting of:
(a) a copolymer having the structure: 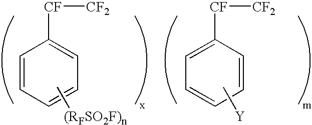
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m and x are mole fractions wherein m is 0.01 to 0.99 and
x is 0.99 to 0.01; and
x+m=1
(b) a copolymer having the structure: 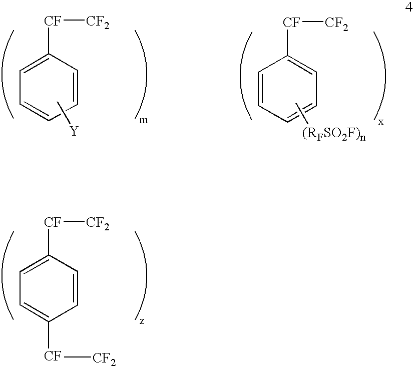
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m, x and z are mole fractions wherein m is 0.01 to 0.99,
x is 0.99 to 0.01, and
z is 0.0001 to 0.10
m+x+z=1.

10. The copolymer of claim 9 wherein RF is selected from the group consisting of (CF₂)ᵣ wherein r=1 to 20, (CF₂CF₂)ᵣOCF₂CF₂ wherein r=0 to 6, and (CF(CF₃)O)ᵣCF₂CF₂ wherein r=1 to 8.

11. The copolymer of claim 10 wherein RF is selected from the group consisting of (CF₂)ᵣ wherein r=1 to 8, (CF₂CF₂)ᵣOCF₂CF₂ wherein r=0 to 2, and CF(CF₃)O)ᵣCF₂CF₂ wherein r=1 to 2.

12. The copolymer of claim 9 wherein the linear or branched perfluoroalkyl and non-fluorinated alkyl groups, wherein the alkyl group comprises C1 to C10 carbon atoms is selected from the group consisting of CₙF₂ₙ₊₁, wherein n is 1 to 10; and CₙH₂ₙ₊₁, wherein n is 1 to 10.

13. The copolymer of claim 9 wherein the perfluoroalkyl group containing oxygen, chlorine or bromine, and wherein the alkyl group comprises C1 to C10 carbon atoms is selected from the group consisting of CF₃(CF₂)_{q}O(CF₂CF₂)_{q} wherein q=1 to 5 and CF₃CF₂CF₂(OCFCF₃)_{q} wherein q=1 to 5.

14. The copolymer of claim 13 wherein the perfluoroalkyl group containing oxygen, chlorine or bromine is selected from the group consisting of CF₃(CF₂)_{q}OCF₂CF₂ wherein q=1 to 2, and CF₃CF₂CF₂(OCFCF₃)_{q} wherein q=1 to 3.

15. The copolymer of claim 14 wherein n is 1.

16. The copolymer of claim 9 wherein m and x are mole fractions, wherein m is 0.1 to 0.4; and x is 0.9 to 0.6 in structure 3.

17. The copolymer of claim 9 wherein m, x and z are mole fractions, wherein m is 0.2 to 0.6; x is 0.4 to 0.8; and z is 0.002 to 0.01 in structure 4.

18. A polymer electrolyte membrane prepared from a homopolymer or copolymer selected from the group consisting of:
(a) a homopolymer having the structure: 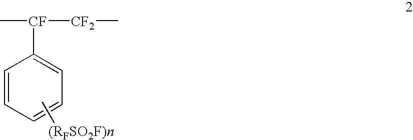
wherein R_{F} is linear or branched perfluoroalkene group, optionally containing oxygen or chlorine,
n is 1 or 2;
(b) a copolymer having the structure: 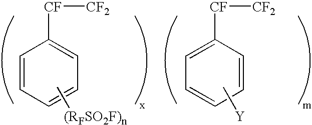
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m and x are mole fractio wherein m is 0.01 to 0.99 and
x is 0.99 to 0.01; and
x+m=1; and
(c) a copolymer having the structure: 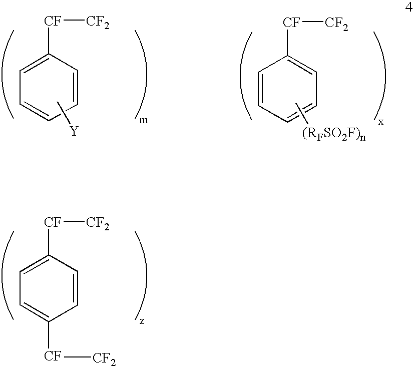
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m, x and z are mole fraction wherein m is 0.01 to 0.99,
x is 0.99 to 0.01, and
z is 0.0001 to 0.10
m+x+z=1; and mixtures thereof.

19. The polymer electrolyte membrane of claim 18 further comprising a porous support.

20.-30. (canceled)

31. A polymer electrolyte membrane selected from the group consisting of:
(a) a membrane having the chemical structure: 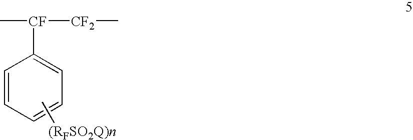
wherein R_{F} is linear or branched perfluoroalkene group, optionally containing oxygen or chlorine,
Q=OM, OH, NHSO2R_{F}, wherein M=Li+, Na, K or Cs,
n=1 or 2;
(b) a membrane having the chemical structure: 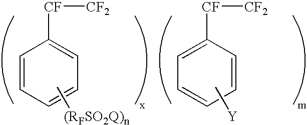
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
Q=OM, OH, NHSO2R_{F}, wherein M=Li⁺, Na⁺, K⁺ or Cs⁺,
n is 1 or 2,
m and x are mole fractions wherein m is 0 to 0.99,
x is 1 to 0.001, and
x+m=1; and
(c) a membrane having the chemical structure: 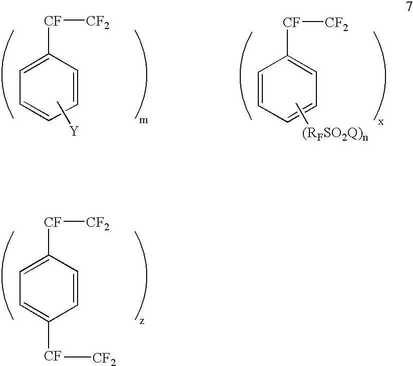
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
Q=OM, OH, NHSO2R_{F}, wherein M=Li⁺, Na⁺, K⁺ or Cs⁺,
n is 1 or 2,
m, x and z are mole fractions wherein m is 0.01 to 0.99
x is 0.99 to 0.01,
z is 0.0001 to 0.10, and
m+x+z=1

32. A membrane electrode assembly comprising a polymer electrolyte membrane, having a first surface and a second surface, wherein the membrane is prepared from a homopolymer or copolymer selected from the group consisting of:
(a) a homopolymer having the structure: 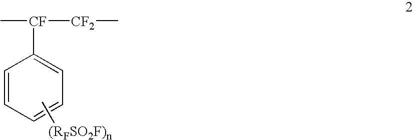
wherein R_{F} is linear or branched perfluoroalkene group, optionally containing oxygen or chlorine,
n is 1 or 2;
(b) a copolymer having the structure: 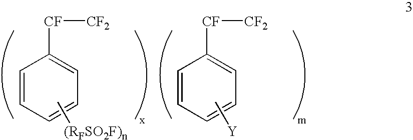
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m and x are mole fractions wherein m is 0.01 to 0.99,
x is 0.99 to 0.01; and
x+m=1; and
(c) a copolymer having the structure: 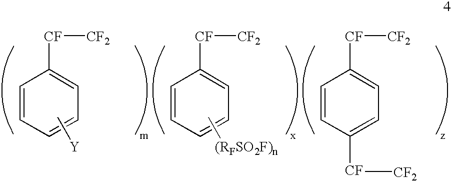
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m, x and z are mole fractions wherein m is 0.01 to 0.99,
x is 0.99 to 0.01, and
z is 0.0001 to 0.10
m+x+z=1; and mixtures thereof.

33. (canceled)

34. The membrane electrode assembly of claim 32 further comprising at least one electrode prepared from an electrocatalyst coating composition present on the first and second surfaces of the membrane.

35. The membrane electrode assembly of claim 34 further comprising at least one gas diffusion backing present on the at least one electrode on the side away from the polymer electrolyte membrane.

36. The membrane electrode assembly of claim 32 further comprising a gas diffusion electrode present on the first and second surfaces of the membrane, wherein the gas diffusion electrode comprises a gas diffusion backing and an electrode prepared from an electrocatalyst containing composition.

37.-47. (canceled)

48. A electrochemical cell comprising a membrane electrode assembly, wherein the membrane electrode assembly comprises a polymer electrolyte membrane, having a first surface and a second surface, wherein the membrane is prepared from a homopolymer or copolymer selected from the group consisting of:
(a) a homopolymer having the structure: 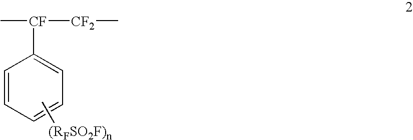
wherein R_{F} is linear or branched perfluoroalkene group, optionally containing oxygen or chlorine,
n is 1 or 2;
(b) a copolymer having the structure: 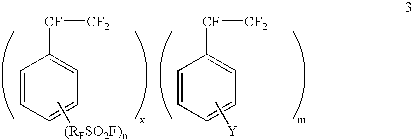
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m and x are mole fractions wherein m is 0.01 to 0.99,
x is 0.99 to 0.01,
x+m=1, and
(c) a copolymer having the structure: 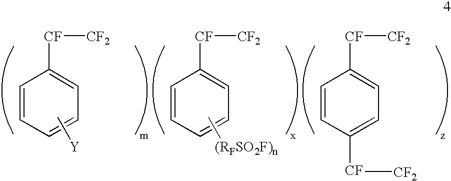
wherein R_{F} is linear or branched perfluoroalkene group, optionally containing oxygen or chlorine,
Y is H; halogen such as Cl, Br, F or I; linear or branched perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms,
n is 1 or 2,
m, x and z are mole fractions wherein m is 0.01 to 0.99,
x is 0.99 to 0.01,
z is 0.0001 to 0.10, and
m+x+z=1; and mixtures thereof.

49. The electrochemical cell of claim 48 wherein the electrochemical cell is a fuel cell.

50.-58. (canceled)
